(19) United States
(12) Patent Application Publication
(10) Pub. No.: US 2026/0309649 A1
(43) Pub. Date: Oct. 8, 2026

(54) RESECTION MARGIN ASSESSMENT

(71) Applicant: Jettec AB, Stocksund (SE)

(72) Inventors: Hans M. Hertz, Stocksund (SE); Jenny Romell, Järfälla (SE); Carlos Fernandez Moro, Hägersten (SE); Filip Lagerqvist, Stockholm (SE)

(21) Appl. No.: 19/476,966

(22) PCT Filed: Apr. 18, 2023
(86) PCT No.: PCT/EP2023/060000

§ 371 Date: Oct. 20, 2025

(51) Int. Cl. G01N 23/046; G01N 23/041; G01N 23/083

(52) U.S. Cl. CPC G01N 23/046; G01N 23/041; G01N 23/083

(57) ABSTRACT

A method for intraoperative assessment of a tumor resection margin in a tissue sample obtained from a patient undergoing surgery for removal of a solid tumor is comprising the steps of: (a) fixing and slicing the tissue sample to provide a flat fixed tissue sample of 0.5-9 mm thickness, wherein the fixing is dehydration for less than 60 minutes in a liquid comprising acetone; and (b) subjecting the flat fixed tissue sample from step (a) to phase-contrast X-ray imaging to generate multiple X-ray projections. (c) obtaining a 3D image of the flat fixed tissue samples by phase-contrast computed tomography (PC-CT) using the X-ray projections obtained in step (b).

### TECHNICAL FIELD OF THE INVENTION

The present invention relates to an improved cancer treatment by surgery. More specifically, the invention relates to a method for intraoperative assessment of a tumor resection margin in a tissue sample obtained from a patient undergoing surgery for removal of a solid tumor.

### BACKGROUND ART

When a patient has a primary tumor, one treatment is to remove the tumor by surgery. The surgeon needs to be completely sure that the entire tumor has been removed during the procedure, since it will require additional treatment otherwise. One way to do this is to evaluate the resection margins, i.e. the margin of non-tumorous tissue surrounding the tumor that has been removed (tumor resection margin assessment), using classical histology. Routine histology involves fixation with formalin, multi-step dehydration with ethanol, infiltration, embedding, sectioning, rehydration and staining to allow for a light-microscopy evaluation. It takes several days to get the result. Classical histology provides great detail and pathological insight via elaborate staining methods in combination with high 2D resolution microscopy. However, it is time consuming (typically several days), requires large amount of manual labor, and does not provide 3D data.

In the classical histopathology, tumor-positive cells are found in the resection margin in 10-35% of the case involving the most common solid cancers. When including also vulvar and oral cancer, the number increase to 40-60%. Typically, this does not lead to renewed surgery, due to risk and patient discomfort. Instead, the post-surgery treatment plan is adjusted, including more chemotherapy and/or radiation therapy.

It is beneficial to use intraoperative (during surgery) feedback to evaluate the tumor resection margin. In frozen section analysis (FSA), the tissue sample is quickly frozen and then analyzed using histology by a pathologist. The freezing of the sample takes about 10 minutes, and the total time of receiving the results takes about 30-60 minutes, which is an acceptable time for intraoperative assessment. Problems with this method are that only 2D images of small tissue biopsies can be acquired, and that the image quality is below the quality given by classical histology. It is very labor intensive.

With improvements to the fixation time of soft tissue, phase-contrast computed tomography (PC-CT) could allow 3D intraoperative feedback to the surgeons. PC-CT is an X-ray method that measures the shift in phase of the X-rays as they pass through the imaged material. This is different to regular X-ray imaging, which measures the attenuation of photons.

The high recurrence rates are disturbing both from a patient outcome perspective and from health economy perspective. Accordingly, there is a need for an improved method for intraoperative assessment of a tumor resection margin in a tissue sample obtained from a patient undergoing surgery for removal of a solid tumor.

### SUMMARY OF THE INVENTION

It is an object of the present invention to provide an improved method for intraoperative assessment of a tumor resection margin in a tissue sample obtained from a patient undergoing surgery for removal of a solid tumor.

It is a further object to provide an improved method for intraoperative assessment of a tumor resection margin in a tissue sample obtained from a patient undergoing surgery for removal of a solid tumor which allows for rapid fixation, which enables intraoperative feedback and therefore improved surgical outcome.

It is an object to provide an improved method for intraoperative assessment of a tumor resection margin in a tissue sample obtained from a patient undergoing surgery for removal of a solid tumor, which achieves improved contrast and/or quality in the images obtained.

It is also an object to provide an improved method for intraoperative assessment of a tumor resection margin in a tissue sample obtained from a patient undergoing surgery for removal of a solid tumor, which achieves improved differentiation between healthy and cancerous tissue in the images obtained.

For these and other objects which will be evident from this disclosure, the present invention provides according to a first aspect a method comprising the steps of:
(a) fixing and slicing the tissue sample to provide flat fixed tissue samples of 0.5-9 mm thickness, wherein the fixing is dehydration for less than 60 minutes in a liquid comprising acetone; and
(b) subjecting the flat fixed tissue samples from step (a) to phase-contrast X-ray imaging to generate multiple X-ray projections.
(c) obtaining a 3D image of the flat fixed tissue samples by phase-contrast computed tomography (PC-CT) using the X-ray projections obtained in step (b).

The inventors have realized and demonstrated that instead of the classical fixation, acetone is surprisingly useful in the intraoperative assessment method as a fixation fluid prior to the phase-contrast X-ray imaging, because it achieves
a rapid fixation of relatively thick tissue samples, which enables intraoperative feedback from PC-CT and therefore improved surgical outcome
generation of contrast without additional agents, such as fluorophores, radionuclides or other biomarkers
improved contrast in the 3D images obtained from the PC-CT
improved differentiation between healthy and cancerous tissue in the 3D images obtained from the PC-CT.

In one embodiment, wherein step (a) is comprising the substeps:
(a1) Slicing the tissue sample into one or more flat tissue samples; and
(a2) Fixing at least one of the flat tissue samples to provide the flat fixed tissue samples.

In an embodiment, the fixing is a single-step dehydration for less than 60 minutes in a liquid comprising acetone.

In one embodiment, the liquid used in step (a) comprises at least 70%, such as at least 75 vol % acetone. In certain embodiments, the liquid used in step (a) comprises at least 80%, such as at least 85 vol % acetone. In some embodiments, the liquid used in step (a) comprises at least 90%, such as at least 95 vol % acetone. In certain embodiments, the liquid used in step (a) is acetone. An increased acetone content increases the speed of the fixing, which contributes to achieve the objective of an intraoperative assessment.

In one embodiment, the phase-contrast computed tomography of step (c) is a micro-computed tomography.

In one embodiment, the method is further comprising a step:
(d) analyzing the PC-CT images to assess whether the tumor resection margin contains cancerous tissue or not.

In an embodiment, the phase-contrast X-ray imaging in step (b) is propagation-based phase-contrast X-ray imaging (PBI).

According to a second aspect, there is provided use of acetone in a fixation liquid to improve the differentiation between non-cancerous and cancerous tissue in an intraoperative phase-contrast X-ray imaging assessment on a phase-contrast computed tomography (PC-CT) image of a tumor resection margin in a tissue sample obtained from a patient undergoing surgery for removal of a solid tumor.

In one embodiment, the fixation liquid comprises at least 70 vol % acetone. In other embodiments, the fixation liquid comprises even higher percentages of acetone as set out above. In certain embodiments, the fixation liquid is acetone.

In an embodiment, the phase-contrast computed tomography is a micro-computed tomography.

In one embodiment, the phase-contrast X-ray imaging is propagation-based phase-contrast X-ray imaging (PBI).

Preferred embodiments and further aspects are defined in the appended claims, listed embodiments and throughout the application text.

### BRIEF DESCRIPTION OF THE DRAWINGS

FIG. 1 shows a comparison of phase-contrast tomography and classical histology in a pancreas sample with a neuroendocrine tumor.

FIG. 2 shows a comparison of formalin- and acetone-fixed tenderloin tissue.

FIGS. 3, 4 and 6 show PC-CT images of three different acetone-fixed colorectal liver metastasis tissues.

FIG. 5 shows PC-CT images of acetone-fixed cholangiocarcinoma tissue.

FIG. 7 shows PC-CT images of acetone-fixed hepatocellular carcinoma tissue.

FIG. 8 shows PC-CT images of acetone-fixed chondrosarcoma tissue.

FIG. 9 shows PC-CT images of acetone-fixed osteosarcoma tissue.

### DETAILED DESCRIPTION

Surgery is an essential part of the curative plan for most patients affected with solid tumors. The outcome of such surgery (recurrence rates and patient survival) depends on several factors where the resection margin is key indicator. The resection margin is the rim of apparently nontumorous tissue around a tumor that has been surgically removed. For a tumor to be considered completely removed, the resection margin should be free of tumorous cells.

All surgical oncology is followed by a histopathological assessment of the resection margin, seeking for tumorous cells. This classical process is presently time-consuming, it typically takes several days before the surgeon receives the pathology report. It is estimated that tumor-positive margins occur in 5%-35% of the cases, depending on cancer type.

Furthermore, due to the present process of slicing, the pathologist risks missing small groups of tumorous cells hidden within the slices. The time, the inadequate margin assessment, and the hidden tumor cells all risk compromising patient outcome.

The present invention provides a method for intraoperative assessment of a tumor resection margin in a tissue sample obtained from a patient undergoing surgery for removal of a solid tumor. Advantageously, the method enables a 3D intraoperative assessment of the tumor resection margin.

As used herein, an intraoperative assessment refers to an assessment which occurs or is performed during the course of a surgical operation. The surgical operation is associated with removal of a solid tumor, partly or preferably completely. The assessment is an objective histopathological determination of the status of a removed tissue sample, in particular to distinguish between nontumorous tissue and tumorous tissue in the tissue sample.

As used herein, a tissue sample refers to a sample of tissue which has been surgically removed from a patient having a solid tumor. The tissue sample may contain the whole solid tumor which has been surgically removed. The tissue sample may also be prepared from the whole solid tumor which has been surgically removed. In particular, the tissue sample encompasses an assumed outer boundary of the solid tumor which has been surgically removed.

As used herein, the tumor resection margin refers to the rim of apparently nontumorous tissue around a solid tumor that has been surgically removed. For a solid tumor to be considered completely removed, the resection margin should be free of tumorous cells. Often it is preferable to keep the margin as small as possible, e.g., for cosmetic reasons (e.g. breast, skin) or to preserve organ function (e.g. liver, kidney).

As used herein, a solid tumor refers to an abnormal mass of tissue that usually does not contain cysts or liquid areas. Solid tumors can occur in several places, including bones, muscles and organs. Solid tumors may be benign or malignant. Different types of solid tumors are named for the type of cells that form them, e.g. sarcomas, carcinomas, and lymphoma. Preferred solid tumor types include liver, pancreas, colon, bone, prostate, breast and skin cancers, but the method is considered as applicable for any solid tumor. As exemplified herein, the method is applicable to liver, pancreas, colon and bone cancers.

Using the method as defined herein, the surgeon can get correct and immediate intraoperative feedback from the pathologist while still having the patient available on the operating table. Such fast intra-operative feedback allows immediate corrective surgery for a more complete surgical tumor removal.

The first method step defines (a) fixing and slicing the tissue sample to provide flat fixed tissue samples of 0.5-9 mm thickness, wherein the fixing is dehydration for less than 60 minutes in a liquid comprising acetone.

The tissue sample is sliced to a thickness of 0.5-9 mm, such as 0.5-7, 0.5-5, 0.5-4, 0.5-3 mm or 1-7, 1-5, 1-4, 1-3 mm or 2-7, 2-5, 2-4, 2-3 mm, e.g. about 3 mm or about 4 mm, to form flat tissue samples. The flat tissue samples are advantageously sliced prior to fixing, such that step (a) is comprising the substeps:
(a1) Slicing the tissue sample into one or more flat tissue samples; and
(a2) Fixing at least one of the flat tissue samples to provide the flat fixed tissue sample.

Alternatively, the flat tissue samples can be prepared by slicing tissue samples which have already been subjected to fixing. In order to achieve sufficient penetration of the fixation liquid in the short time span, it is then advantageous to have a tissue sample which has a thickness of less than 30 mm, such as less than 20 mm, preferably less than 15 mm or less than 10 mm. It is also possible to slice the tissue samples into flat tissue samples during the actual fixing, i.e. at the same time as the dehydration occurs.

As defined herein, the fixing is occurring for less than 60 minutes, such as 20-60 min, 30-60 min or 40-60 min. In this step, the tissue sample, which has preferably been sliced into flat tissue samples of 0.5-9 mm thickness, is subjected to dehydration for less than 60 minutes in a liquid comprising acetone.

Preferably, the fixing is a single step dehydration occurring for less than 60 minutes, such as 20-60 min, 30-60 min or 40-60 min. In this single step, the tissue sample, which has preferably been sliced into flat tissue samples of 0.5-9 mm thickness, is subjected to dehydration for less than 60 minutes in a liquid comprising acetone.

The inventors have realized that instead of the classical fixation, acetone is useful as a fixation fluid prior to phase-contrast X-ray imaging. Compared to the classical fixation, the advantages are:
Quicker fixation, which enables intraoperative feedback from phase-contrast X-ray imaging and therefore improved surgical outcome
Generation of contrast without additional agents, such as fluorophores, radionuclides or other biomarkers
Improved contrast in the images obtained from the phase-contrast X-ray imaging
Improved differentiation between healthy and cancerous tissue in the images obtained from the phase-contrast X-ray imaging.

It is particularly surprising that the acetone liquid itself generates sufficient contrast, and that no additional step and/or additional agent is necessary to achieve the contrast needed for differentiation between non-cancerous and cancerous tissue in the X-ray images.

The goal with fixating tissue is traditionally to preserve morphological features in the tissue. The fixation process therefore allows for dissection of the tissue without movement. By eliminating these movements, examination of the tissue at hand can be performed without much structural differences in the tissue. Structural differences are here referred to as changes when comparing the tissue in its natural environment and the tissue when it has been resected. The present inventors have realized that a rapid fixation using acetone will advantageously result in a differential shrinkage of the treated tissue. In traditional histology, differential shrinkage has been highly undesirable, and complicated methods have been developed to avoid differential shrinkage so as to preserve morphological features in the tissue. Without wishing to be limited to any particular theory, it is considered that for the present purpose of rapidly distinguishing between healthy and cancerous tissue (rather than providing a morphologically optimal picture), the differential shrinkage, e.g. between healthy and cancerous tissue, obtained with rapid acetone fixation provides a sufficient contrast in the phase-contrast X-ray imaging.

For the purpose of rapidly distinguishing between healthy and cancerous tissue and determining tumor resection margin, the present method involving rapid fixation with acetone provides improved contrast and/or differentiation, both compared to rapid fixation without acetone and compared to light microscopy pictures using classical histology. Compared to classical histology, an improved differentiation is achieved with the present method due to the creation of a 3D image.

To achieve a rapid dehydration, the fixing liquid of step (a) is comprising at least 70 vol % (vol/vol) acetone. Preferably, the fixing liquid of step (a) is comprising at least 75 vol % acetone, such as at least 80 vol % acetone, such as at least 85 vol % acetone, such as at least 90 vol % acetone, such as at least 95 vol % acetone, or even at least 99 vol % acetone. The remainder of the fixing liquid may be water and/or any other suitable liquid, e.g. ethanol, methanol, acetic acid, trichloroacetic acid, picric acid, trinitrophenol, zinc chloride, zinc acetate, chloral hydrate, glyoxal, or xylene, or combinations thereof.

Examples of compositions which are useful as fixing liquids include, without limitation thereto:
70-99 vol % acetone, 1-30 vol % ethanol;
80-99 vol % acetone, 1-20 vol % ethanol;
90-99 vol % acetone, 1-20 vol % ethanol;
70-99 vol % acetone, 1-30 vol % methanol;
80-99 vol % acetone, 1-20 vol % methanol;
90-99 vol % acetone, 1-10 vol % methanol;
70-99 vol % acetone, 1-30 vol % acetic acid;
80-99 vol % acetone, 1-20 vol % acetic acid; and
90-99 vol % acetone, 1-10 vol % acetic acid.

The fixing liquid may also comprise formaldehyde or glutaraldehyde. In certain embodiments, formaldehyde and glutaraldehyde are excluded from the fixing liquid.

Preferably, the fixing liquid does not contain any water.

In a preferred embodiment, the fixing liquid of step (a) is acetone, i.e. 100 vol % acetone.

The tissue sample may be treated with the fixation liquid at room temperature or above room temperature. A higher temperature provides a more rapid fixation step, and it is therefore preferred that the fixation temperature is 20-60° C., preferably 30-50° C., such as 40-50° C.

Advantageously, the flat fixed tissue samples obtained from step (a) do not require any further embedding step, e.g. in paraffin. If desired, it is possible to include a further embedding step, e.g. a paraffin embedding step, between steps (a) and (b).

The second method step defines (b) subjecting the flat fixed tissue samples from step (a) to phase-contrast X-ray imaging. Thereby, multiple X-ray projections are generated. As used herein, phase-contrast X-ray imaging or phase-sensitive X-ray imaging comprise technical methods that use information concerning changes in the phase of an X-ray beam that passes through an object in order to create images. The beam's phase shift caused by the sample is not measured directly, but is transformed into variations in intensity, which then can be recorded by a detector. Advantageously, several/all flat fixed tissue samples can be imaged simultaneously.

Phase contrast allows detailed X-ray imaging of soft tissues, where classical absorption contrast does not provide sufficient contrast. The 3D phase contrast images obtained include both absorption contrast and phase contrast. This has obvious clinical and pre-clinical value, as demonstrated for e.g. lungs, cartilage, and tumors. To date, most phase-contrast bioimaging aimed at high spatial resolution with cellular detail has been performed at synchrotron light sources since only this high-brightness source provides sufficient coherence and flux to allow phase-contrast imaging with adequate exposure times. Hemberg et al., Appl. Phys. Lett. 83:1483 (2003) developed a liquid-metal-jet electron-impact microfocus source which operates at an order of magnitude higher brightness than competing laboratory sources and with potential for another order of magnitude. This small-spot and high-power X-ray tube enables phase-contrast X-ray imaging with high spatial resolution and adequate exposure times in the clinic.

Examples of useful phase-contrast X-ray imaging techniques include crystal interferometry, propagation-based imaging, analyzer-based imaging, edge-illumination and grating-based imaging. They are all based on the observation of interference patterns between diffracted and undiffracted waves.

In a preferred embodiment, the phase-contrast X-ray imaging in step (b) is propagation-based phase-contrast X-ray imaging (PBI). PBI typically detects the transverse Laplacian of the phase (∇²φ) with a single measurement and without extra optical elements. With a high-brightness and high-spatial-coherence liquid-metal-jet microfocus source, PBI provides a cellular spatial resolution with sufficient contrast at reasonable exposure times. Accurate marking of cellular details (like the resection margin) is achieved with post-processing, including phase retrieval, artifact reduction and tomographic reconstruction. Compared to other methods, PBI results in the shortest exposure times but phase retrieval is necessary for quantitative interpretation. Exposure time and resolution are important parameters. As a comparison, for imaging gas-filled structures (like CO₂-filled blood vessels or air-filled lung alveoli) the necessary exposure time for observing structures smaller than 50 μm may differ a factor ten in favor of PBI, see e.g. Zhou et al., Opt. Express. 21, 30183 (2013) . . . . The method is readily expandable to 3D imaging via tomographic methods, see e.g., U. Lundström et al., Phys Med. Biol. 57, 2603-2617 (2012).

Phase-contrast X-ray tomography allows cellular-resolution 3D imaging of the resection margin in tumors. Advantageously, the pathological assessment is done in full 3D (three dimensions) with cellular resolution, to capture all cellular-sized pathological features, not only those on top of the slice. Furthermore, phase contrast X-ray imaging is more sensitive to density variations in the sample than conventional transmission-based X-ray imaging. This leads to images with improved soft tissue contrast.

It is particularly surprising that the acetone liquid itself generates sufficient contrast, and that no additional step and/or additional agent is necessary to achieve the contrast needed for differentiation between non-cancerous and cancerous tissue in the X-ray images.

The third method step defines (c) obtaining a 3D image of the flat fixed tissue samples by phase-contrast computed tomography (PC-CT) using the X-ray projections obtained in step (b). Step (c) comprises obtaining a phase-contrast computed tomography (PC-CT) 3D image of the flat fixed tissue samples from the X-ray data obtained in step (b). As used herein, tomography refers to imaging by sections or sectioning that uses a penetrating wave, in this case X-rays. The phase contrast provides a better definition of the edges between tumor and healthy tissue. The phase contrast X-ray imaging data obtained in step b) is combined with tomographic techniques to obtain a 3D distribution of the real part of the refractive index of the sample. The X-rays are used to create cross-sections of a physical object that can be used to recreate a virtual model (3D model) without destroying the original object. As phase contrast X-ray imaging is more sensitive to density variations in the sample than conventional transmission-based X-ray imaging, images with improved soft tissue contrast can be obtained.

Compared to conventional histology, phase-contrast X-ray tomography on excised samples provide advantages including speed, 3D imaging with thinner effective slicing, and less destructive and simplified sample preparation. For intraoperative resection margin assessment, high spatial resolution and short exposure times are essential. For the purposes of intraoperative assessment of a tumor resection margin in a tissue sample obtained from a patient undergoing surgery for removal of a solid tumor, it is realized that a short exposure time is required but at the same time a sufficiently high resolution to differentiate between non-cancerous and cancerous tissue. Previously, histology with similar resolution and exposure time as in the present resection margin project has only been observed at synchrotron radiation facilities, on rat brain specimen (Töpperwien et al., Sci Rep. 7, 42847 (2017)) and human coronary arteries (Holme et al., Nature Prot. 9, 1401 (2014)). However, for our application, the local access close to the operating suite is a must, ruling out the synchrotron-source-based methods.

The method preferably utilizes a high-resolution X-ray tomography and more specifically X-ray microtomography, implying that the pixel sizes of the cross-sections are in the micrometer range. In practice, all tomography today is computed tomography. Consequently, the method step c) preferably utilizes micro-computed tomography (sometimes abbreviated micro-CT or μCT).

Micro-CT has applications both in medical imaging and in industrial computed tomography. In general, there are two types of scanner setups. In one setup, the X-ray source and detector are typically stationary during the scan while the sample/animal rotates. The second setup, much more like a clinical CT scanner, is gantry based where the animal/specimen is stationary in space while the X-ray tube and detector rotate around. These scanners are typically used for small animals (in vivo scanners), whereas for biomedical samples, foods, microfossils, and other studies for which minute detail is desired, the X-ray source and detector are kept stationary.

An exemplary system includes a metal-jet x-ray source (Excillum D2+, 250 W), a high-accuracy rotating stage, and a large high-resolution detector, all on an approximately 200×80 cm radiation-safe bench. The sample holder provides simple, fast, safe, and flexible sample handling. The key factors for observing the relevant low-contrast cellular-sized details at short exposure times is optimizing source parameters, distances and detector parameters. This system has demonstrated 1 μm resolution in 3D imaging. By exchanging the source to a 1 kW source, the acquisition times can be reduced a factor 4.

The power of the x-ray source is preferably at least 100 W, more preferably at least 200 W.

The software integrates hardware control and calibration, scan algorithms, tomographic reconstruction, and image processing algorithms. High-fidelity imaging relies on the synergy of hardware scan algorithms and post-data-acquisition image reconstruction. Key techniques involve phase retrieval, reduction of reconstruction artifacts, mathematical noise filtering, and observability analysis.

The method may further comprise a step (d) of analyzing the PC-CT image to assess whether the tumor resection margin contains cancerous tissue or not.

As a consequence of the assessment in step (d), it may be concluded that the surgery has been sufficient. Alternatively, it may be decided to resume surgery and excise more tissue from the patient, in which case the method for intraoperative assessment may be repeated.

According to a second aspect, there is also provided a novel use of acetone in a fixation liquid to improve the differentiation between non-cancerous and cancerous tissue in an intraoperative phase-contrast X-ray imaging assessment on a phase-contrast computed tomography (PC-CT) image of a tumor resection margin in a tissue sample obtained from a patient undergoing surgery for removal of a solid tumor.

The inventors have realized that instead of the classical fixation, acetone is useful as a fixation fluid prior to phase-contrast X-ray imaging. Compared to the classical fixation, the advantages are:
Quicker fixation, which enables intraoperative feedback from phase-contrast X-ray imaging and therefore improved surgical outcome
Generation of contrast without additional agents, such as fluorophores, radionuclides or other biomarkers
Improved contrast in the images obtained from the phase-contrast X-ray imaging
Improved differentiation between healthy and cancerous tissue in the images obtained from the phase-contrast X-ray imaging.

It is particularly surprising that the acetone liquid itself generates sufficient contrast, and that no additional step and/or additional agent is necessary to achieve the contrast needed for differentiation between non-cancerous and cancerous tissue in the X-ray images.

To achieve a rapid dehydration, the fixing liquid of step (a) is comprising at least 70 vol % (vol/vol) acetone. Preferably, the fixing liquid of step (a) is comprising at least 75 vol % acetone, such as at least 80 vol % acetone, such as at least 85 vol % acetone, such as at least 90 vol % acetone, such as at least 95 vol % acetone, or even at least 99 vol % acetone. The remainder of the fixing liquid may be water and/or any other suitable liquid, e.g. ethanol, methanol, acetic acid, trichloroacetic acid, picric acid, trinitrophenol, zinc chloride, zinc acetate, chloral hydrate, glyoxal, or xylene, or combinations thereof.

Examples of compositions which are useful as fixing liquids include, without limitation thereto:
70-99 vol % acetone, 1-30 vol % ethanol;
80-99 vol % acetone, 1-20 vol % ethanol;
90-99 vol % acetone, 1-20 vol % ethanol;
70-99 vol % acetone, 1-30 vol % methanol;
80-99 vol % acetone, 1-20 vol % methanol;
90-99 vol % acetone, 1-10 vol % methanol;
70-99 vol % acetone, 1-30 vol % acetic acid;
80-99 vol % acetone, 1-20 vol % acetic acid; and
90-99 vol % acetone, 1-10 vol % acetic acid.

The fixing liquid may also comprise formaldehyde or glutaraldehyde. In certain embodiments, formaldehyde and glutaraldehyde are excluded from the fixing liquid.

Preferably, the fixing liquid does not contain any water.

In a preferred embodiment, the fixing liquid of step (a) is acetone, i.e. 100 vol % acetone.

In connection to the advantageous use of acetone in the fixation liquid, the method preferably utilizes a high-resolution X-ray tomography and more specifically X-ray microtomography, implying that the pixel sizes of the cross-sections are in the micrometer range.

Examples of useful phase-contrast X-ray imaging techniques include crystal interferometry, propagation-based imaging, analyzer-based imaging, edge-illumination and grating-based imaging. They are all based on the observation of interference patterns between diffracted and undiffracted waves.

In a preferred embodiment, the phase-contrast X-ray imaging in step (b) is propagation-based phase-contrast X-ray imaging (PBI).

The present invention will in the following be further illustrated by the following non-limiting examples.

### EXAMPLES

### Example 1—Phase-Contrast X-Ray Histology

### System

The instrument includes a metal-jet X-ray source (Excillum D2+, 250 W), a high accuracy rotating stage, and a large high-resolution detector, all on an approximately 200×80 cm radiation-safe bench. The sample holder provides simple, fast, safe, and flexible sample handling. The key factors for observing the relevant low-contrast cellular-sized details at short exposure times is optimizing source parameters, distances and detector parameters.

The software integrates hardware control and calibration, scan algorithms, tomographic reconstruction, and image processing algorithms in a user-friendly package. High-fidelity imaging relies on the synergy of hardware scan algorithms and post-data-acquisition image reconstruction. Key techniques involve phase retrieval, reduction of reconstruction artifacts, mathematical noise filtering, and observability analysis. The system, supplied by Exciscope AB, has demonstrated 1 μm resolution in 3D imaging.

### Results—Tumor Resection Margin Imaging

We have tested our phase-contrast X-ray histology on formalin-fixed and paraffin-embedded slices from pancreas and liver cancers retrospectively, i.e., after the classical “top-layer” histology has been performed at a hospital laboratory. The samples were then investigated with X-ray optical equipment and mathematical tools. This methodology has the advantage that the phase-contrast X-ray imaging can be compared directly to the “top-layer” classical histology on the same samples, while still no disturbance is done to the clinical procedure.

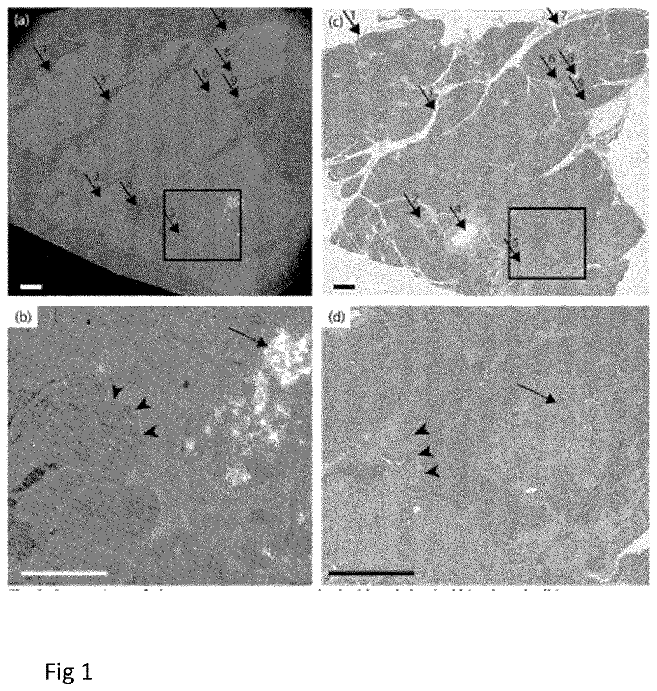

FIG. 1 shows a comparison of phase-contrast tomography (a, b) and classical histology (c,d) in a pancreas sample with a neuroendocrine tumor. The tumor (bottom right a and c) has lower density than the healthy tissue, and therefore appears darker in the x-ray image. This sample is rich in microcalcifications, appearing as bright spots. The phase-contrast tomography (a, b) shows good correspondence to the histology (c,d), even though the contrast mechanisms are fundamentally different. We note that the tumor and resection margin are clearly observable. Data looks equally good for ductal adenocarcinoma (liver) and cystic neoplasm (benign).

### Example 2—Fixation Procedure-Animal Samples

Fixation of tenderloin meat with acetone and formalin was compared and visually evaluated based on contrast with PC-CT.

### X-Ray Imaging Arrangement

A micro-CT system based on a MetalJet D2 X-ray source (Excillum AB, Stockholm, Sweden) was used. The acceleration voltage of this source was set to 70 kV and the power was set to 100 W. The distances R1 (X-ray source to object) and R2 (object to detector) were set to 350 mm and 550 mm, respectively.

With this configuration, acquisition of 3001 X-ray images was made with the tissue rotating 360 degrees in total. Along with these 3001 images, eight flat field images were acquired: four images before and four images after the scan. The flat field images were images without the object in the field of view (FOV). The flat field images are necessary in the next step of the PC-CT pipeline, phase retrieval.

### Phase Retrieval

The phase retrieval was performed in MATLAB using a code based on Paganin's algorithm. The phase retrieval acts as a low-pass filter and it allows for a transformation of the sharp edges between two different materials in the projected images, that have different refractive indices, into projected thickness of the sample. In this algorithm, the images taken from the X-ray arrangement were used as input data. With this code sinograms were created and these are necessary in the next step of the PC-CT pipeline, tomographic reconstruction.

### Tomographic Reconstruction

The tomographic reconstruction was performed by the Feldkamp-Davis-Kress (FDK) algorithm using Octopus Reconstruction software. The reconstruction was using the sinograms acquired from the phase retrieval part as input data.

### Sample Preparation

The object in this case was fixed tissue of tenderloin meat. There were two scans, one of tissue fixed in 100% acetone (VWR International S.A.S., Fontenay-sous-Bois, France) and another of tissue fixed in formalin (Formaldehyde solution 4%, buffered, pH 6.9, Merck KGaA, Damstadt, Germany). The tissues were approximately 12×12×12 mm in size, and they were put in a plastic tube with acetone during scanning. The tissue was fixed for approximately 24 hours before scanning took place.

### Results

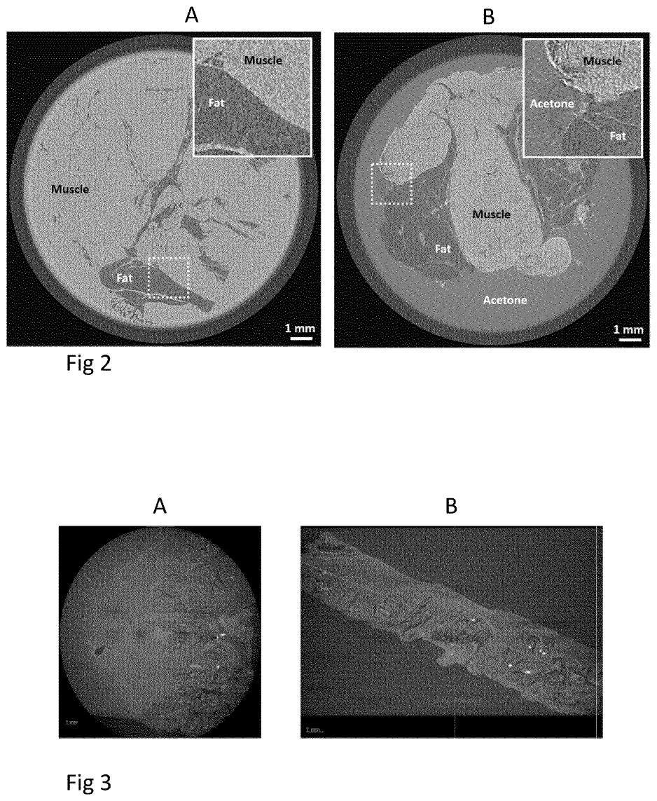

FIG. 2 shows a comparison of acetone- and formalin-fixed tenderloin tissue. The left image (A) is tenderloin in formalin and the right image (B) is tenderloin in acetone. The insets show enlarged views of interfaces between fat and muscle in the two samples, clearly showing the increased contrast arising in the acetone-fixed sample, where we for example can distinguish individual muscle cells. Overall, the acetone-fixed tissue shows a higher level of detail and higher contrast between different structures within the tissue. In the formalin-fixed tissue sample, only fat and muscle tissue can be clearly distinguished due to their natural difference in density.

### Example 3—Fixation Procedure-Human Samples

### Sample Preparation

The system used in Example 1 was tested on fresh tumors, directly from surgery.

Samples were prepared by immersion in acetone.

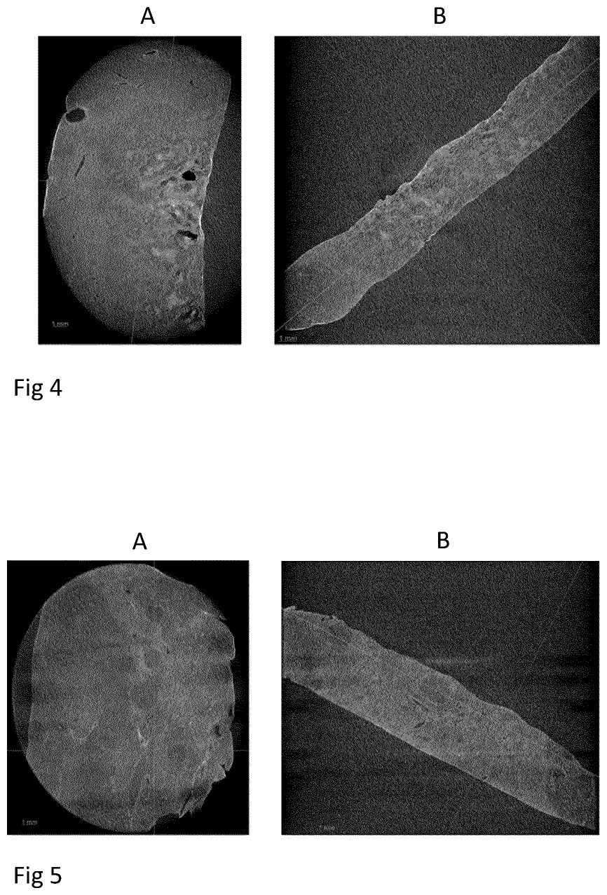

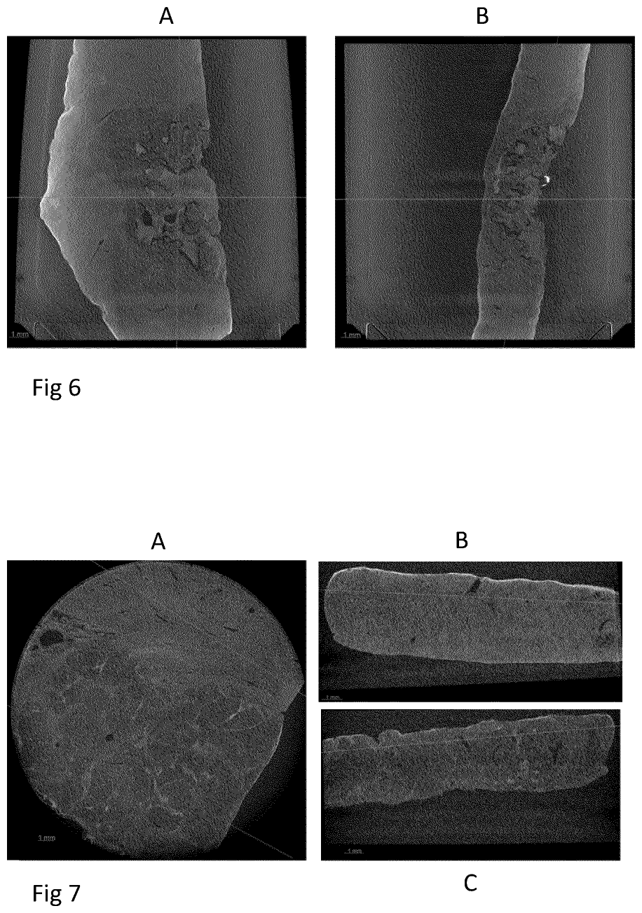

The system was applied on the rapidly fixed resection specimens, directly from surgery, see Table 1 and FIG. 3-7.

TABLE 1
Samples of human cancerous tissue

| | Sample | | |
|---|---|---|---|
| FIG. | size (mm) | Tumour type | Description |
| 3A-B | 20 × 25 × 5 | Colorectal liver | Colon cancer spread |
| | | metastasis | to the liver |
| 4A-B | 15 × 25 × 5 | Colorectal liver | Colon cancer spread |
| | | metastasis | to the liver |
| 5A-B | 15 × 20 × 5 | Cholangiocarcinoma | Bile duct cancer |
| 6A-B | 10 × 20 × 5 | Colorectal liver | Colon cancer spread |
| | | metastasis | to the liver |
| 7A-C | 20 × 20 × 5 | Hepatocellular | Most common type of |
| | | carcinoma | primary liver cancer, |
| | | | starting in hepatocytes |

Further imaging is performed with focus on samples from liver, pancreas, and colon cancers. Results are evaluated by comparing our X-ray histology with the classical histology for the same samples. The evaluation is performed by expert pathology observers, independent and blinded, for proper statistical analysis.

### Example 4—Fixation Procedure-Human Samples

### Sample Preparation

The system used in Example 1 was tested on fresh tumors, directly from surgery.

Samples were prepared by immersion in acetone.

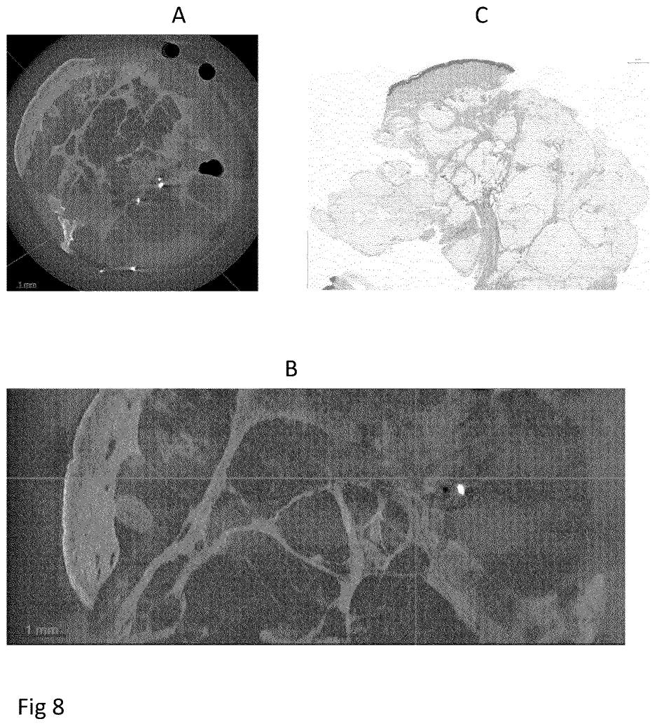

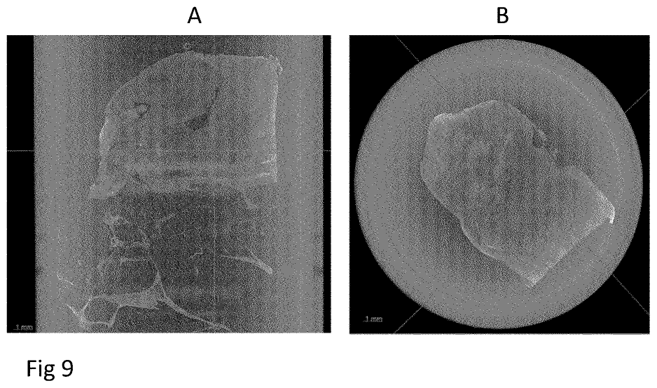

The system was applied on the rapidly fixed resection specimens, directly from surgery, see Table 2 and FIG. 8-9.

TABLE 2
Samples of human cancerous tissue

| | Sample | | |
|---|---|---|---|
| | diameter | Tumour | |
| FIG. | (mm) | type | Description |
| 8A-C | 12 | Chondrosarcoma | Bone cancer, origin |
| | | | in cartilage cells |
| 9A-B | 12 | Osteosarcoma | Bone cancer, origin |
| | | | in bone cells |

1. A method for intraoperative assessment of a tumor resection margin in a tissue sample obtained from a patient undergoing surgery for removal of a solid tumor, comprising the steps of:
(a) fixing and slicing the tissue sample to provide flat fixed tissue samples of 0.5-9 mm thickness, wherein the fixing is dehydration for less than 60 minutes in a liquid comprising acetone;
(b) subjecting the flat fixed tissue samples from step (a) to phase-contrast X-ray imaging to generate multiple X-ray projections-; and
(c) obtaining a 3D image of the flat fixed tissue samples by phase-contrast computed tomography (PC-CT) using the multiple X-ray projections obtained in step (b).

2. The method of claim 1, wherein step (a) is comprising the substeps:
(a1) slicing the tissue sample into one or more flat tissue samples; and
(a2) fixing at least one of the one or more flat tissue samples to provide the flat fixed tissue samples.

3. The method of claim 1, wherein the fixing is a single-step dehydration for less than 60 minutes in a liquid comprising acetone.

4. The method of claim 1, wherein the liquid used in step (a) comprises at least 70 vol % acetone.

5. The method of claim 4, wherein the liquid used in step (a) comprises at least 80 vol % acetone.

6. The method of claim 5, wherein the liquid used in step (a) is acetone.

7. The method of claim 1, wherein the phase-contrast computed tomography of step (c) is a micro-computed tomography.

8. The method of claim 1, further comprising a step:
(d) analyzing the 3D image to assess whether the tumor resection margin contains cancerous tissue or not.

9. The method of claim 1, wherein the phase-contrast X-ray imaging in step (b) is propagation-based phase-contrast X-ray imaging (PBI).

10-16. (canceled)

17. The method of claim 1, wherein the tissue sample of step (a) is obtained from the patient undergoing surgery for removal of the solid tumor.

18. A method for intraoperative assessment of a tumor resection margin in a tissue sample obtained from a patient undergoing surgery for removal of a solid tumor, comprising the steps of:
(a) fixing and slicing the tissue sample obtained from the patient undergoing surgery for removal of the solid tumor to provide flat fixed tissue samples of 0.5-9 mm thickness, wherein the fixing is dehydration for less than 60 minutes in a liquid comprising acetone;
(b) subjecting the flat fixed tissue samples from step (a) to phase-contrast X-ray imaging to generate multiple X-ray projections; and
(c) obtaining a 3D image of the flat fixed tissue samples by phase-contrast computed tomography (PC-CT) using the multiple X-ray projections obtained in step (b);
the method thereby improving differentiation between non-cancerous and cancerous tissue on a phase-contrast computed tomography (PC-CT) image of the tumor resection margin in the tissue sample.

19. The method of claim 18, wherein step (a) is comprising the substeps:
(a1) slicing the tissue sample into one or more flat tissue samples; and
(a2) fixing at least one of the one or more flat tissue samples to provide the flat fixed tissue samples.

20. The method of claim 18, wherein the fixing is a single-step dehydration for less than 60 minutes in a liquid comprising acetone.

21. The method of claim 18, wherein the liquid used in step (a) comprises at least 70 vol % acetone.

22. The method of claim 21, wherein the liquid used in step (a) comprises at least 80 vol % acetone.

23. The method of claim 22, wherein the liquid used in step (a) comprises at least 90 vol % acetone.

24. The method of claim 23, wherein the liquid used in step (a) is acetone.

25. The method of claim 18, wherein the phase-contrast computed tomography of step (c) is a micro-computed tomography.

26. The method of claim 18, further comprising a step:
(d) analyzing the 3D image to assess whether the tumor resection margin contains cancerous tissue or not.

27. The method of claim 18, wherein the phase-contrast X-ray imaging in step (b) is propagation-based phase-contrast X-ray imaging (PBI).
